(19) United States
(12) Patent Application Publication
(10) Pub. No.: US 2021/0169378 A1
(43) Pub. Date: Jun. 10, 2021

(54) METHOD FOR PROCESSING AN ACCELEROMETRIC SIGNAL

(71) Applicant: Université Grenoble Alpes, Saint Martin d'Heres (FR)

(72) Inventors: Grégoire Gerard, Tassin La Demi Lune (FR); Pierre-Yves Gumery, Grenoble (FR); Damien Colas, Caluire-et-Cuire (FR); Aurélien Bricout, Lyon (FR)

(21) Appl. No.: 17/052,480

(22) PCT Filed: Apr. 30, 2019
(86) PCT No.: PCT/FR2019/051010

§ 371 Date: Nov. 2, 2020

(30) Foreign Application Priority Data
May 2, 2018 (FR) 1853796

(51) Int. Cl. A61B 5/113; G16H 40/67; G16H 50/30; A61B 5/00; A61B 5/11; G01P 15/18

(52) U.S. Cl. CPC A61B 5/1135; G16H 40/67; G16H 50/30; A61B 5/6823; A61B 2562/0219; A61B 5/725; A61B 5/1116; G01P 15/18; A61B 5/7221

(57) ABSTRACT

A signal-processing method comprises storing, in memory, a first initial signal generated by a first set of at least one three-axis accelerometer positioned in a thoracic position of an individual. A second initial signal, generated by a second set of at least one three-axis accelerometer in an abdominal position of the individual, is also stored in the memory. The second initial signal is synchronised with the first initial signal. The data of the first initial signal is processed to compute a first final vector, representing thoracic forces experienced by the first set of at least one three-axis accelerometer. The data of the second initial signal is processed to compute a second final vector, representing abdominal forces experienced by the second set of at least one three-axis accelerometer.

### CROSS-REFERENCE TO RELATED APPLICATIONS

This application is a national phase entry under 35 U.S.C. § 371 of International Patent Application PCT/FR2019/051010, filed Apr. 30, 2019, designating the United States of America and published as International Patent Publication WO 2019/211561 A1 on Nov. 7, 2019, which claims the benefit under Article 8 of the Patent Cooperation Treaty to French Patent Application Serial No. 1853796, filed May 2, 2018.

### TECHNICAL FIELD

The present disclosure relates to the field of monitoring of the respiration (or breathing) of an individual.

More precisely, it relates, in the field of well-being and health, to the measurement and characterization of the breathing function of an individual by virtue of a set of networked accelerometers.

The present disclosure allows data relating to the respiration of an individual to be collected. Subsequent analysis of these data may, for example, be used to obtain a characterization of a breathing motion or a medical diagnosis.

The present disclosure, is, for example, applicable to:
sleep analysis and research into breathing-related sleep disorders,
sport and the exploration of respiratory functions, in particular with regard to the individual's aerobic sporting performance but also for training to free dive,
public well-being, for example for the evaluation of respiratory dynamics in a static position,
monitoring respiratory dynamics during relaxing activities such as yoga,
analysis of the response of an individual to a given stress, for example to a video campaign, a film, a piece of music, etc.
the field of clinical and pre-clinical research, for the evaluation and validation of the effect of certain drugs on respiratory functions, and
the field of general and hospital-based medicine, for the rapid screening of respiratory pathologies.

### BACKGROUND

Currently, respiratory-plethysmography bands, which are placed around the chest of an individual, are employed. However, such bands are impractical to use because they may move and/or deteriorate during their use, this then making their data inexploitable, and their intrinsic fragility means they must be handled with care.

It would therefore appear to be desirable to improve this existing solution.

### BRIEF SUMMARY

More precisely, the present disclosure relates to a signal-processing method, comprising or consisting of:
storing beforehand in a memory a first initial signal generated by a first set of at least one three-axis accelerometer (110) positioned on the thorax of an individual.

It is essentially characterized in that it furthermore comprises:
storing beforehand in a memory a second initial signal generated by a second set of at least one three-axis accelerometer (120) positioned on the abdomen of the individual, the second signal being synchronized with the first signal,
processing the data of the first initial signal to compute a first final vector, representative of the thoracic efforts undergone by the first set of at least one three-axis accelerometer, and
processing the data of the second initial signal to compute a second final vector, representative of the abdominal efforts undergone by the second set of at least one three-axis accelerometer.

Preferably, the step of processing the data of the first initial signal and the step of processing the data of the second initial signal comprise a step of detecting a time window of instability in the first initial signal and a step of detecting a time window of instability in the second initial signal, and, in case of detection of a time window of instability, the method then furthermore comprises a step of temporarily inhibiting the computation of the first final vector and a step of temporarily inhibiting the computation of the second final vector, preferably until a time window of stability is regained, respectively.

Provision may be made for the data of the first initial signal and the data of the second initial signal to be stored in the form of a first matrix and in the form of a second matrix, respectively; this being followed by at least one of the steps among:
processing the data of the first initial signal, and
processing the data of the second initial signal,

comprising a step of principal component analysis of the vectors of the first matrix and a step of principal component analysis of the vectors of the second matrix to obtain the first final vector and to obtain the second final vector, respectively,

the method preferably furthermore comprising a step of filtering the first final vector.

Provision may furthermore be made for a step comprising or consisting of computing at each measurement time the rotation angle with respect to gravity of the first set and second set, respectively, of at least one three-axis accelerometer to obtain the first final vector and to obtain the second final vector, respectively.

Provision may be made for an initial pre-processing step comprising or consisting of under-sampling the signals of each channel of each accelerometer.

Provision may be made for a step of applying a band-pass filter to at least one among the first signal and the second signal, the band-pass filter preferably having a lower cut-off frequency equal to 0.05 Hz and an upper cut-off frequency equal to 0.8 Hz, and optionally being a finite-impulse-response filter.

Provision may be made for a step comprising or consisting of qualifying the overall respiratory effort of the individual.

Preferably, the qualification of effort comprises a linear combination of the first final vector and of the second final vector.

The qualification of effort may also comprise a step comprising or consisting of computing, for at least one among the first set (110) and the second set (120) of at least one three-axis accelerometer, a normalized cross-correlation, to characterize the periodic character of the respiratory effort.

Provision may also be made for the qualification of effort to comprise a step comprising or consisting of computing at least one of the descriptors among:
any phase shift between the signal generated by the first set of at least one accelerometer and the signal generated by the second set of at least one accelerometer,
the coupling between the signal generated by the first set of at least one accelerometer and the signal generated by the second set of at least one accelerometer, as given by intercorrelation or mutual cross-information, and
an index of thoracic-abdominal desynchronization.

Provision may also be made for a step of detecting a change in position of the individual.

According to the present disclosure, the pair of accelerometers alone allows the equivalent of a respiratory inductance plethysmograph (RIP) to be provided. In this sense, the present disclosure may be considered to be a virtual respiratory inductance plethysmograph because it is composed at least solely of two accelerometers, one placed on the thorax of the subject and the other on the abdomen of the subject.

The present disclosure is especially of advantage in the miniaturization of the measurement system, which is less complex, less expensive, unintrusive and less invasive that the standard systems used in hospitals, and in its ability to provide a reliable, robust and rapid measurement.

Other features and advantages of the present disclosure will become more clearly apparent on reading the following description, which is given by way of illustrative and non-limiting example, with reference to the accompanying figures.

### BRIEF DESCRIPTION OF THE DRAWINGS

FIG. 1 illustrates a set of six raw signals obtained from three channels of a thoracic accelerometer and of an abdominal accelerometer, and a reference signal obtained via an abdominal plethysmographic band and a reference signal obtained via an abdominal plethysmographic band.

FIG. 2 synchronously illustrates an experimental signal PHI and a reference signal PRI, the signals being obtained using the signals of FIG. 1.

FIG. 3 illustrates the concordance between the two signals PHI and PRI of FIG. 2, according to a Bland-Altman plot.

FIG. 4 synchronously illustrates a signal dPHI (derivative of the signal PHI of FIG. 2) a signal dPRI (derivative of the signal PRI of FIG. 2) and a reference signal FLOW.

FIG. 5 synchronously illustrates a signal sPHI and a signal sPRI.

FIG. 6 illustrates the computation of an index characterizing the periodicity of a thoracic breathing effort.

FIG. 7 illustrates the computation of an index characterizing the periodicity of another thoracic breathing effort.

FIG. 8A illustrates one embodiment of a measuring device according to the present disclosure.

FIG. 8B illustrates another embodiment of a measuring device according to the present disclosure.

### DETAILED DESCRIPTION

For the sake of brevity, by:
“thoracic accelerometer” or “first accelerometer,” what is meant is a first set of at least one three-axis accelerometer positioned on the thorax of the subject,
“abdominal accelerometer” or “second accelerometer,” what is meant is a second set of at least one three-axis accelerometer positioned on the abdomen of the subject,
“individual,” “patient,” or “subject,” what is meant is any natural person whether they be healthy or suffering from any pathology,
“signals” or “data,” what is meant is the values measured along each axis of the first and second accelerometer, and
“compartment,” what is meant is the thorax or the abdomen.

Measuring Device:

A single and compact measuring device comprising at least two accelerometers configured to measure acceleration along three axes is provided.

In particular, provision is made for a first set of at least a first accelerometer, which is intended to be placed on the thorax of the individual, preferably on his xiphoid process, or position.

Provision is also made for a second set of at least one accelerometer, which is intended to be placed on the abdomen of the individual.

Each set of at least one accelerometer may comprise a plurality of accelerometers, for example for reasons of redundancy and robustness.

For the sake of brevity, the first set of at least one accelerometer is referred to as the first accelerometer 110, and the second set of at least one accelerometer is referred to as the second accelerometer 120.

In the present case, a single accelerometer per set may be sufficient, i.e., a single first accelerometer placed on the thorax and a single second accelerometer on the abdomen.

Each accelerometer emits a respective signal comprising a set of data and called the “initial signal,” which corresponds to the variation in the accelerations (in g) as a function of time. For the sake of brevity, the terms “signal” and “data” have been used interchangeably.

Preferably, provision is made for the measuring device to comprise analog or digital and optionally wireless means for activating/deactivating the accelerometers, these means for example taking the form of an activation control switch.

In a first variant, provision is made for the first accelerometer and the second accelerometer to be securely fastened to a single holder, or support 100 (FIG. 8A).

In a second variant, provision is made for the first accelerometer and the second accelerometer to be securely fastened to respective holders, or supports 200, 210 (FIG. 8B).

Whatever the variant, each holder, or support, has an upper face and a lower face. The upper face bears at least one accelerometer and the lower face, which is intended to make contact with the individual, is typically coated with an adhesive (glue) that is preferably repositionable. The advantage of the repositionable aspect is that the individual may remove the measuring device on waking, after a shower, etc. and then reuse the same measuring device, and do so over a number of consecutive days. The device is, for example, powered by a battery.

The first accelerometer and the second accelerometer may be identical. In the present case, the first accelerometer and the second accelerometer are standard three-axis accelerometers.

The data output from each sensor are stored in a computer memory. They are sent by wire or wirelessly.

In a first variant, the memory is connected by a computer cable to the first and the second accelerometers. The memory may, for example, be placed on the single holder, or support, or on any one of the holders, or supports, of the accelerometers.

Preferably, provision is made for the memory to be computationally connected to an input/output (I/O) communication port, a USB port, for example, this allowing the stored data to be exported to a data-processing device comprising a data-processing software package, this device typically being any communicating object, i.e., an electronic device comprising wired or wireless communication means, a processor and preferably a display screen, for example, a personal computer, a smartphone, a touchscreen tablet, etc.

Provision may also be made for the memory to be removable, for example, in the form of a data medium such as a memory card with a USB connector, that is connectable to the first accelerometer and to the second accelerometer with a view to storing the data obtained therefrom, and then connectable to a computer equipped with a filtering software package allowing the data to be filtered.

In a second variant, the memory is a remote memory, i.e., one without a wired connection between the memory and the sensors. For example, the memory is located remotely in the data-processing device.

In this case, provision is advantageously made for the measuring device to comprise means for communicating with the data-processing device, this communication being either by wire or wireless.

The memory may therefore be a local memory, or a remote memory located on a server, in particular, of a cloud computing system.

The accelerometers are advantageously mounted on a circuit board comprising a battery, a processor (microcontroller) and a storage unit capable of storing the data with a view to local processing. Alternatively, each accelerometer is mounted on an independent circuit board, each having a battery for supplying power, a processor and a storage unit. Provision is made for the optional sensors to be securely fastened to the circuit board of the first set of at least one accelerometer. The signals are synchronized, by design, by the microcontroller.

Preferably, means are also provided for processing the signals generated by the sensors.

In the present case, the signal-processing means comprise filtering means configured to filter the initial signal of the first accelerometer, and configured to filter the initial signal of the second accelerometer.

Similarly to memory, the filtering means may be integrated into the measuring device, or located remotely in the data-processing device or even on a server in communication with the data-processing device.

In this case, provision is advantageously made for the measuring device to comprise means for communicating with the signal-processing means, this communication being either by wire or wireless.

Each three-axis accelerometer possesses three orthonormal channels (or axes) called X, Y and Z, respectively, the angular position of which with respect to gravity must be determined.

By convention:
by X1, Y1 and Z1, what is meant is the three channels of the first accelerometer, and
by X2, Y2 and Z2, what is meant is the three channels of the second accelerometer.

It will be noted that once the first accelerometer and the second accelerometer have been positioned on an individual, the axes X1, Y1 and Z1 are not necessarily coincident with the axes X2, Y2 and Z2, respectively.

For each accelerometer, the sampled data are stored in the form of a starting matrix with three columns, one column for the X-axis, one column for the Y-axis, and one column for the Z-axis, the matrix containing as many rows as there are sampling points; for example, L=60*T*F with L the total number of rows, T the sampling time in minutes and F the sampling frequency.

There is therefore a starting matrix for the thoracic compartment and a starting matrix for the abdominal compartment.

The individual's respiration generates movements of his rib cage and/or of his abdomen. The first accelerometer and the second accelerometer allow, as described below, these movements to be reconstructed, in particular via computation of their respective angular velocities.

As the XYZ axes are orthonormal, knowing the position of two axes among X, Y and Z with respect to gravity allows the other to be reconstructed. Furthermore, when the placement of the accelerometer on the individual is defined by convention, this allows the individual/accelerometer frame of reference to be set. Therefore, in this case knowledge of the position of a single axis among X, Y and Z with respect to gravity allows the two others to be reconstructed.

When an individual breathes, the movement of the rib cage or abdomen is very slight. Consequently, the speed of movement, and therefore the acceleration, is negligible. The signal generated by an accelerometer is therefore not really, to the first order, related to the movement of the rib cage or to the movement of the abdomen.

In contrast, when an individual breathes, the (thoracic or abdominal) accelerometers undergo a rotation with respect to gravity. When an accelerometer pivots, the gravity vector also pivots in the frame of reference X1Y1Z1 of the first accelerometer and in the frame of reference X2Y2Z2 of the second accelerometer.

The present disclosure aims to follow this pivoting motion, and to compute the variation in the orientation of the gravity vector in the reference frame X1Y1Z1 or X2Y2Z2 over time.

Preferably, the individual is considered to remain static on the whole during the recording of the data output from the accelerometers, so as to make the signals stationary, which makes it possible to prevent the measurements of the accelerometers from being affected by noise due to the movement of the individual.

As described below, the principle of the present disclosure comprises or consists of measuring, according to a reference axis, the variations in inclination of the accelerometers with respect to gravity each time the stationarity of the signal changes, then in processing these signals in order to reconstruct the breathing of the individual.

Pre-processing:

Provision is made for a pre-processing step, comprising or consisting in under-sampling the signals obtained from each channel of the accelerometers.

Preferably, provision is made for the signals originating from the first accelerometer and the signals originating from the second accelerometer to be sampled at the same sampling frequency Fe.

Preferably, provision is made for the sampling frequency Fe to be lower than a preset threshold value, so as to under-sample the signals.

For example, Fe=256 Hz.

Preferably, provision is also made for the sampling frequency Fe to be higher than another preset threshold value. For example, provision is made for the Nyquist frequency=Fe/2 to be higher than the high cut-off frequency multiplied by a reference value, in the present case 10, so as to obtain a Nyquist frequency at least equal to 8 Hz.

Filtering:

After the pre-processing step, provision is made for a step of filtering the signals.

Provision is made to filter each channel of the accelerometers with the same filter. Preferably, provision is made for a band-pass filtering. It is possible to make provision for a low cut-off frequency of between 0.01 Hz and 0.1 Hz and a high cut-off frequency of between 0.6 Hz and 1 Hz. In the present case, provision is made for a finite-impulse-response (FIR) band-pass filter of low cut-off frequency equal to 0.05 Hz and of high cut-off frequency equal to 0.8 Hz.

Reconstruction:

After the filtering step, provision is made for a step of reconstructing respiratory efforts, i.e., the movements behind the signals generated by the (thoracic) first accelerometer and the signals generated by the (abdominal) second accelerometer, according to at least one of the two variants below.

Preferably, each of the two variants below is implemented after each detection of a change in the position of the individual.

1^{st} Variant: Principal Component Analysis (PCA):

Provision may be made for a step of principal component analysis of each starting matrix, for each compartment, and thus to decrease the size of each matrix.

In the present case, this makes it possible to pass from an L*3 matrix to an L*1 vector.

For the data generated by a given accelerometer, a principal component analysis of these data is a projection into a new space, i.e., a linear combination of each initial variable, in a new space, that describes the maximum variance in the XYZ frame of reference of the accelerometer.

Thus, at each time, the PCA allows the axis X1, Y1 or Z1 of the first accelerometer and the axis X2, Y2 or Z2 of the second accelerometer that describes the maximum variance (at least 80%) and that pivots with respect to the axis of gravity to be determined.

The previous steps will have allowed noise of physiological origin (for example, cardiac activity) and various artefacts (movements, noises, etc.) to be filtered (removed) from the accelerometer signals while preserving respiratory activity, or in other words from the measurement of the pivoting motion of the first and second accelerometers. Therefore, the axis that exhibits the maximum variability is the one that conveys the most respiration-related information.

In the present case, for each accelerometer, the sampled data are stored in the form of a starting matrix with three columns, one column for the X-axis, one column for the Y-axis, and one column for the Z-axis, the matrix containing as many rows as there are sampling points; for example, L=60*T*F with L the total number of rows, T the sampling time in minutes and F the sampling frequency.

The PCA comprises or consists of converting the starting matrix into an end vector each coefficient of which is a respective linear combination of the corresponding starting-matrix row, and therefore of the three axes of the starting matrix.

In the present case, the PCA comprises or consists of measuring the eigenvalues and eigenvectors of the variance-covariance matrix of the starting matrix. The eigenvector with the highest eigenvalue is considered to be the principal component and will serve as a vector for the projection into a new space in which each coordinate is a linear combination of each (starting) axis (X, Y and Z).

It is then possible to compute the vector A_Respi resulting from the multiplication of the starting matrix A_PCA by the principal component CP:

$$CP = [ \begin{matrix}α \\ β \\ γ\end{matrix} ]$$

$${A}_{PCA} = [ \begin{matrix}X 1 & Y 1 & C 1\end{matrix} ]$$

$${A}_{Respi} = {A}_{PCA} * CP = [ \begin{matrix}X 1 & Y 1 & Z 1\end{matrix} ] * [ \begin{matrix}α \\ β \\ γ\end{matrix} ]$$

Each coordinate of the resulting vector A_Respi is therefore, with as an example the coordinate i:

$${A}_{Respi} ( i ) = {A}_{PCA} ( i , : ) * CP = [ \begin{matrix}X 1 ( i ) & Y 1 ( i ) & Z 1 ( i )\end{matrix} ] * [ \begin{matrix}α \\ β \\ γ\end{matrix} ] = X 1 ( i ) * a Y 1 ( i ) * β Z 1 ( i ) * γ$$

Thus the following are obtained:
a first final vector, resulting from a principal component analysis of a first starting matrix corresponding to the data generated by the first accelerometer, and
a second final vector, resulting from a principal component analysis of a second starting matrix corresponding to the data generated by the second accelerometer.

Each final vector is a reconstruction: the first final vector is a reconstruction of the respiratory effort of the thoracic compartment and the second final vector is a reconstruction of the respiratory effort of the abdominal compartment.

However, PCA is a method that is sensitive to noise. The filtering according to the present disclosure described above allows noise to be decreased. However, it may furthermore be advantageous to detect certain noise-generating events, for example a change in the position of the individual.

To this end, provision may be made to measure the energy of the signal of the first and/or second accelerometer in a set of moving time windows, preferably two adjacent time windows that overlap partially.

A thresholding step comprising or consisting of comparing, in each time window, the energy of the signal of the first and/or the second accelerometer with a preset threshold value is then provided, and if the energy of the signal is greater than the preset threshold value, then the data of this time window are not taken into account in the PCA (i.e., in the measurement of the eigenvector serving as basis for the projection into the new space).

Final Filtering:

Provision may then be made for a step of applying a final filter to the final vector.

Preferably, the final filter is the same as the band-pass filter described above.

2^{nd} Variant: Angular Method:

It may also be advantageous, instead of or in addition to the PCA method, to implement a method referred to as the angular method.

In this variant, the aim is to compute the angle of rotation of each accelerometer with respect to gravity at each measurement time.

To this end, provision is made to measure and normalize the acceleration vector, which is denoted a.

Under normal conditions, the user is static and the accelerations due to respiration are negligible. Therefore, under normal conditions, the acceleration vector a is substantially equal to the acceleration due to gravity g.

When an accelerometer pivots, the gravity vector also pivots in the intrinsic frame of reference of the accelerometer. Such a pivoting motion is due to respiration and therefore varies over time. Therefore, the angular movement of the accelerometer may be considered to take place along a single rotation axis r.

It is therefore a question of measuring the angular movement of the rotation axis r of the accelerometer.

The rotation angle θt and the rotation axis rt of the acceleration vector a between two consecutive measurements at time t−1 and at time t are, respectively, given by the scalar product and the cross product of the following two vectors:

θₜ=cos⁻¹(aₜ·aₜ₋₁)

rₜ=aₜ×aₜ₋₁

Respiration is an oscillatory movement. Therefore, the rotation axis r of the acceleration vector a inverts when the direction of rotation inverts. It would therefore appear to be useful to normalize this direction of the rotation axis r in a preset hemisphere, by comparison with a reference axis r_{ref}.

It is thus possible to define:

$${r}_{t}^{′} = \{ \begin{matrix}{r}_{t} , & {r}_{t} \cdot {r}_{ref} \geq 0 \\ - {r}_{t} , & {r}_{t} \cdot {r}_{ref} < 0\end{matrix}$$

Preferably, in order to decrease the influence of noise and to increase the influence of measurements taken close to time t, provision is made to determine r̅ₜ, the normalized rotation axis r averaged over a time window of duration W such that:

$${\overline{r}}_{t} = normalize ( {\sum }_{i = - W / 2}^{W / 2} H ( i ) {θ}_{t + 1} {r}_{t + i}^{′} )$$

with H(n) a Hamming window. Other windows may be used, for example, a rectangular window, a triangular window, a Hann window, a Blackman window, a Kaiser window, etc.

Similarly, provision is made to determine āₜ, the normalized average acceleration averaged over a time window of duration W, preferably the same time window as for r̅ₜ, such that:

$${\overline{a}}_{t} = normalize ( {\sum }_{i = - W / 2}^{W / 2} {a}_{t + 1} )$$

It is then possible to compute ϕₜ, the rotation angle of the accelerometer such that:

ϕₜ=sin⁻¹((āₜ×r̅ₜ)·aₜ)

It is thus possible to compute, if necessary, the angular velocity ωt, such that:

$${ω}_{t} = \frac{d φ}{dt}$$

At least the values of the angle of rotation ϕₜ are stored in the form of a final vector.

Virtual RIP:

Thus, irrespectively of whether the first variant, the second variant, or a combination of the two variants is employed, a first final vector that qualifies thoracic effort and a second final vector that qualifies abdominal effort are obtained, this allowing a virtual RIP to be obtained.

By virtue of this first final vector and this second final vector, it is possible to qualify the overall respiratory effort of the individual, i.e., the relative volume and the time-domain dynamics of the efforts of each compartment, and therefore to qualify their coherence in the time domain.

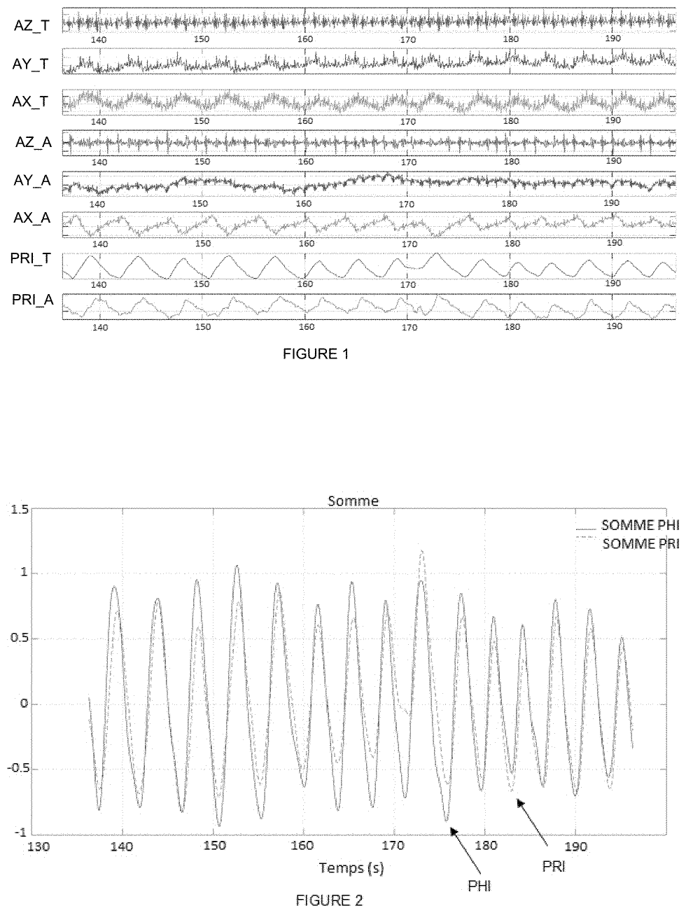

FIG. 1 synchronously illustrates:
the raw data AX_T, AY_T and AZ_T generated by the first accelerometer along the XYZ axes of the first accelerometer;
the raw data AX_A, AY_A and AZ_A generated by the second accelerometer, along the XYZ axes of the second accelerometer; and
the raw data PRI_T and PRI_A of a plethysmography band positioned on the thorax and on the abdomen, respectively.

The thoracic RIP (PRI_T) and the abdominal RIP (PRI_A) illustrated in FIG. 1 are variations in cross-sectional area and serve as a reference.

The sum of the thoracic RIP (PRI_T) and the abdominal RIP (PRI_A) is an RIP signal that provides an image of tidal volume.

FIG. 2 illustrates the normalized superposition between the values −1 and +1 of an experimental signal PHI and a reference signal PRI.

The reference signal PRI is the sum of the raw data PRI_T and PRI_A of FIG. 1.

The signal PHI is computed, in the present case using the angular method, from the raw data AX_T, AY_T and AZ_T generated by the first accelerometer and from the raw data AX_A, AY_A and AZ_A generated by the second accelerometer.

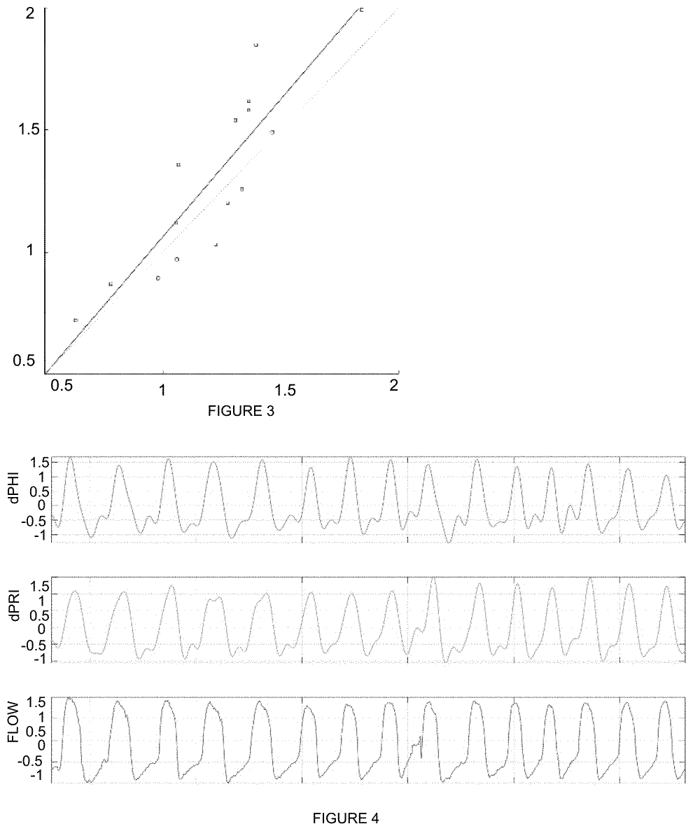

The relevance of the present disclosure is clearly illustrated by FIG. 2. The two signals PHI and PRI are almost always in phase. In the present case, the concordance between the two signals PHI and PRI may be established, for example using a Bland-Altman plot (see FIG. 3), for which a value of r²=0.78 is obtained.

Reconstruction of an Image of Volume:

There is a correspondence between the angular velocity of a thoracic and abdominal accelerometer and the waveform of the overall respiratory breathing effort.

It is therefore possible to provide a step of computing the angular velocity of each accelerometer, this allowing an image of the respiratory flow rate, and the dynamics of the respiratory movements of the individual, to be reconstructed.

To this end, provision is made for at least one of the linear combinations among:
a linear combination of the PCA of the (thoracic) first accelerometer and the PCA of the (abdominal) second accelerometer; and
a linear combination of the angular method applied to the (thoracic) first accelerometer and the angular method applied to the (abdominal) second accelerometer.

FIG. 4 synchronously illustrates:
the signal dPHI, which is the derivative with respect to time of the signal PHI of FIG. 2,
the signal dPRI, which is the derivative with respect to time of the signal PRI of FIG. 2, and
a reference signal FLOW, which is an actual flow-rate signal, in the present case measured by means of a breathing mask placed on an individual.

FIG. 4 also clearly illustrates the relevance of the present disclosure since the computation of the derivative does not give rise to any particular noise. The three signals dPHI, dPRI and FLOW are in phase on the whole. The present disclosure allows an image of the relative volume, and its variation as a function of time, to be computed. In this sense, it is a virtual RIP.

The first accelerometer and the second accelerometer function as a sensor array. Their respective measurements are synchronized and correspond to the acceleration of the thoracic compartment and of the abdominal compartment, respectively.

It is thus possible to compute the respective angular velocities. Next, it is possible to compare the variations in the angular velocity of the first accelerometer and the variations in the angular velocity of the second accelerometer in the same time basis, and to detect, for example, whether the first accelerometer and the second accelerometer are out of phase or in phase opposition, and to compute, for example, an index of abdominal-thoracic desynchronization.

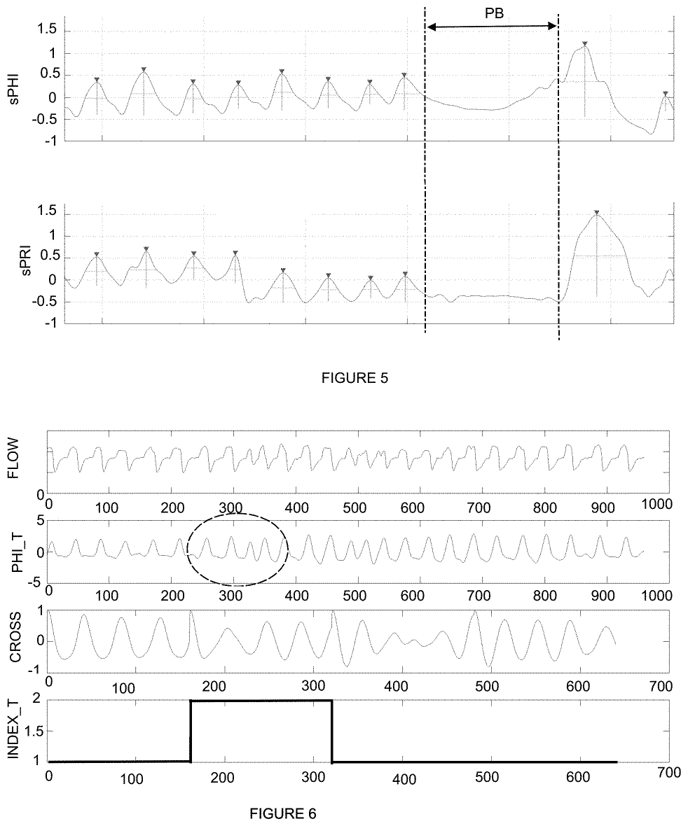

Similarly, FIG. 5 synchronously illustrates a signal sPHI, which is the sum of the signals PHI in the present case of FIG. 1, and a signal sPRI, which is the sum of the signals PRI in the present case of FIG. 1.

It is clear from FIG. 5 that a modification of the respiratory rate (region PB) is detected by the method according to the present disclosure.

The present disclosure allows a “volume image” to be reconstructed for the respiratory compartments and events of respiratory interest to be monitored. It is robust both algorithmically and physiologically.

As explained above, the first variant (PCA) and the second variant (angular method) may be combined.

For example, provision may be made, in a first phase, to implement the first variant (PCA) in a first time window on the measurements of at least one of the accelerometers among the first accelerometer and the second accelerometer, then to implement the second (angular) variant in a time sub-window of the first window.

Qualification of Overall Respiratory Effort:

By virtue of the present disclosure, it is possible, using a single pair of accelerometers, to qualify the breathing mode of operation of an individual.

In a first variant, provision is made to compute a linear combination of the first final vector and the second final vector. For example, provision is made for unit coefficients in the linear combination.

In a second variant, provision is made to compute at least one of the descriptors among:
a normalized cross-correlation of at least one of the final vectors, in order to characterize the periodicity of the respiratory effort with a view to characterizing the periodicity (or non-periodicity) of at least one of the signals among the signal generated by the first accelerometer and the signal generated by the second accelerometer;
   and, for example, to compare the periodicity (or non-periodicity) of one of the two signals generated by the first accelerometer and second accelerometer with respect to the other of the two signals generated by the first accelerometer and second accelerometer,
the difference between the first final vector and the second final vector, to compute at least one among:
   any phase shift between the signal generated by the first accelerometer and the signal generated by the second accelerometer,
   the coupling between the signal generated by the first accelerometer and the signal generated by the second accelerometer, as given by intercorrelation or mutual cross-information,
   an index of thoracic-abdominal desynchronization, and
   for example, to thus qualify the coherence of the thoracic compartment and the abdominal compartment, and
a quantification of the contribution of each compartment to the overall breath.

Instabilities:

In addition, a step may be provided of detecting instabilities, or abnormal, non-stationary for example, events that may, for example, correspond to a change in the position of the individual and/or body movements, that may occur including at night.

Thus each signal generated by the first accelerometer and second accelerometer comprises time windows of stability and potentially a set of at least one time window of instability.

Preferably, provision is made to implement the computations described above in a window of stability.

To this end, provision may be made to compare the value of the signal of at least one of the accelerometers with a preset threshold value, and, for each time window in which the value of the signal is higher than the preset threshold value, to then not take the data of this time window into account in the computations; thus the computations are carried out only in stationary phases during which the individual is immobile, this improving the signal-to-noise ratio.

It is then possible to launch a new sequence of steps of pre-processing, filtering, computing final vectors by PCA/angular method and qualifying the overall respiratory effort.

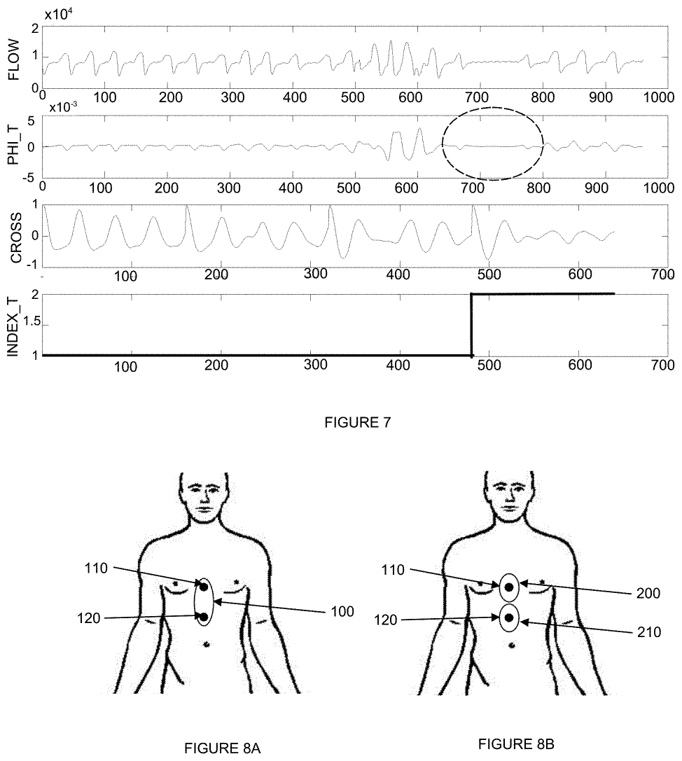

For example, FIG. 6 and FIG. 7 synchronously illustrate:
a reference signal FLOW, which is an actual flow-rate signal, in the present case measured by means of a breathing mask placed on an individual,
a signal PHI_T, which is the signal PHI computed, in this case using the angular method, from the raw data AX_T, AY_T and AZ_T generated by the first accelerometer,
a signal CROSS, which is the normalized cross-correlation PHI_T, and
an index INDEX_T, which is the characterization of the periodicity of the reconstructed thoracic breathing effort PHI_T, the index INDEX_T being a binary signal for which the value 1 corresponds to a periodic signal PHI_T, and the value 2 to a non-periodic signal PHI_T, this index in the present case being obtained by comparing the value of the signal CROSS with a preset threshold value.

In FIG. 6 and in FIG. 7, the signal PHI_T exhibits a periodicity defect, this defect being circled by a dashed circle in FIGS. 6 and 7 (also observable in the reference FLOW).

As illustrated by the signal INDEX_T, this periodicity defect is clearly detected by the method according to the present disclosure (rising edge of the signal INDEX_T in FIG. 6 and FIG. 7).

A return to normal, i.e., a signal PHI_T that becomes periodic again, is also detected by virtue of the method according to the present disclosure, as illustrated by the falling edge of the signal INDEX_T in FIG. 6.

1. A signal-processing method, comprising:
storing, in memory, a first initial signal generated by a first set of at least one three-axis accelerometer positioned on the thorax of an individual;
storing, in the memory, a second initial signal generated by a second set of at least one three-axis accelerometer positioned on the abdomen of the individual, the second initial signal being synchronized with the first initial signal;
processing data of the first initial signal to compute a first final vector, representative of thoracic efforts undergone by the first set of at least one three-axis accelerometer; and
processing data of the second initial signal to compute a second final vector, representative of abdominal efforts undergone by the second set of at least one three-axis accelerometer.

2. The signal-processing method of claim 1, wherein:
processing the data of the first initial signal and processing the data of the second initial signal comprise:
detecting a time window of instability in the first initial signal; and
detecting a time window of instability in the second initial signal; and
the signal-processing method further comprises, after detecting the time window of instability in any of the first initial signal or the second initial signal:
temporarily inhibiting the computation of the first final vector; and
temporarily inhibiting the computation of the second final vector.

3. The signal-processing method of claim 1, wherein:
storing the first initial signal and storing the second initial signal comprise storing data of the first initial signal and storing data of the second initial signal in the form of a first matrix and in the form of a second matrix, respectively; and
at least one of the processing acts comprises a principal component analysis of the first matrix and a principal component analysis of the second matrix to obtain the first final vector and to obtain the second final vector, respectively.

4. The signal-processing method of claim 1, further comprising computing, at each measurement time, a rotation angle with respect to gravity of the first set of at least one three-axis accelerometer and a rotation angle with respect to gravity of the second set of at least one three-axis accelerometer to obtain the first final vector and to obtain the second final vector, respectively.

5. The signal-processing method of claim 1, further comprising applying a band-pass filter to at least one of the first initial signal or the second initial signal.

6. The signal-processing method of claim 1, further comprising qualifying an overall respiratory effort of the individual.

7. The signal-processing method of claim 6, further comprising a linear combination of the first final vector and of the second final vector.

8. The signal-processing method of claim 6, further comprising computing, for at least one of the first set or the second set of at least one three-axis accelerometer, a normalized cross-correlation, to characterize a periodic character of the overall respiratory effort of the individual.

9. The signal-processing method of claim 6, further comprising computing at least one descriptor among the following descriptors:
any phase shift between the first initial signal generated by the first set of at least one three-axis accelerometer and the second initial signal generated by the second set of at least one three-axis accelerometer,
a coupling between the first initial signal generated by the first set of at least one three-axis accelerometer and the second initial signal generated by the second set of at least one three-axis accelerometer, as given by intercorrelation or mutual cross-information, or
an index of thoracic-abdominal desynchronization.

10. The signal-processing method of claim 1, further comprising detecting a change in position of the individual.

11. The signal-processing method of claim 2, wherein the temporary inhibition of the computation of the first final vector and the temporary inhibition of the computation of the second final vector are continued until a time window of stability is regained.

12. The signal-processing method of claim 3, further comprising filtering the first final vector.

13. The signal-processing method of claim 5, wherein applying the band-pass filter comprises applying a band-pass filter having a lower cut-off frequency equal to 0.05 Hz and an upper cut-off frequency equal to 0.8 Hz.

14. The signal-processing method of claim 5, wherein applying the band-pass filter comprises applying a finite-impulse-response filter.
